(19) United States
(12) Patent Application Publication
(10) Pub. No.: US 2024/0285279 A1
(43) Pub. Date: Aug. 29, 2024

(54) DEVICES, SYSTEMS, AND METHODS FOR DELIVERING A DEVICE BETWEEN ANATOMICAL STRUCTURES

(71) Applicant: BOSTON SCIENTIFIC SCIMED, INC., MAPLE GROVE, MN (US)

(72) Inventors: John Thomas Favreau, Shrewsbury, MA (US); Richard Kim, Chestnut Hill, MA (US); Gian Franco Loo Fuchs, San Jose (CR); Yeison Calvo, San Antonio (CR)

(73) Assignee: BOSTON SCIENTIFIC SCIMED, INC., MAPLE GROVE, MN (US)

(21) Appl. No.: 18/584,097

(22) Filed: Feb. 22, 2024

Related U.S. Application Data
(60) Provisional application No. 63/447,726, filed on Feb. 23, 2023.

(51) Int. Cl. A61B 17/11; A61B 17/34; A61F 2/95

(52) U.S. Cl. CPC A61B 17/1114; A61B 17/3468; A61F 2/95; A61B 2017/3456; A61B 2017/3484

(57) ABSTRACT

An access and delivery device and system configured to facilitate access and delivery of an implantable device to a deployment site. The system includes a tissue-penetrating element and an access and delivery device with an anchor member configured to flex outwardly transverse to the tissue-penetrating element. The system may pass through a proximal non-adherent anatomical structure to a deployment site in a distal non-adherent anatomical structure. The anchor member may be used to maintain the relative positions of the non-adherent anatomical structures and/or to protect the deployment site from a tissue-penetrating end of an implantable device delivery and deployment system. The tissue-penetrating element may have a soft distal tip configured to engage tissue walls atraumatically, such as to protect a tissue wall from a sharp tissue-penetrating end of the tissue-penetrating element.

### CROSS-REFERENCE TO RELATED APPLICATIONS

This application claims the benefit of priority under 35 U.S.C. § 119 to U.S. Provisional Application No. 63/447,726, filed Feb. 23, 2023, the entire disclosure of which is hereby incorporated by reference herein for all purposes.

### FIELD

The present disclosure relates generally to the field of devices, systems, and methods for holding anatomical structures with respect to each other and/or moving anatomical structures into apposition with each other. Additionally, the present disclosure relates generally to the field of devices, systems, and methods for delivering and deploying devices across adjacent anatomical structures and protecting tissue of the anatomical structures from tissue-penetrating ends of tissue-penetrating elements.

### BACKGROUND

Complex endoscopic ultrasound (EUS) guided procedures (such as in the gastrointestinal tract) require adequate and accurate positioning while maintaining clear ultrasound imaging in order to perform a procedure safely and effectively to the desired access site. Such procedures may include various tasks, including, without limitation, cutting, cauterizing, delivering a treatment device, etc., which typically require adequate and accurate positioning and control. Although endoscopic imaging modalities, such as fluoroscopy and endoscopic ultrasound (EUS), have the ability to view anatomical structures beyond the proximate, visually accessible, tissue in front of the endoscope, therapeutic endoscopic procedures may be limited by the difficulty of controlling tissue beyond the tissue plane visually accessible to the endoscope. Different anatomical sites pose unique challenges, such as with positioning, distance, and shape. For example, placing a stent between two non-adherent anatomical structures (e.g., tissue walls) such as in a gastrojejunostomy, hepaticogastrostomy, or gallbladder drainage into either the stomach or duodenum is technically challenging due to the lack of tools to visualize, stabilize, and, in some cases, inflate the target site. For instance, once a site is accessed, physicians need to be able to maintain the access without the proximal or distal structure moving away from or slipping with respect to the proximal structure, which may result in losing the access site. For instance, advancement of a stent between two non-adherent anatomical structures may push the distal non-adherent anatomical structure away from the proximal non-adherent anatomical structure. In some instances, the distal site cannot readily be re-accessed if it moves away from the proximal structure, leaving the patient with a perforation in the distal structure and potentially harmful leakage of bodily fluid therefrom. Moreover, the space within a distal site may be limited, and access to such site via a proximal wall of such distal site may be accompanied by the risk of perforating the distal wall of such distal site. All of these challenges can lead to a failed procedure with serious complications in a patient population already in extremely poor health where there is no room for failure. Improvement to delivery and deployment of devices across adjacent anatomical structures, and which optionally hold the anatomical structures in apposition, would be welcome in the field.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an access and delivery device and system is configured to access a deployment site beyond a proximal tissue wall of an anatomical structure. In some embodiments, the access and delivery device and system includes a tissue-penetrating element having a distal end configured to penetrate through tissue walls; and an access and delivery device having a proximal portion, an anchor member, and a distal tip. In some embodiments, the anchor member is formed of a flexible material different from the material of at least the proximal portion of the flexible elongate element, the flexible material configured to flex transversely with respect to the tissue-penetrating element to form an anchor feature of the system.

In some embodiments, the anchor member is formed of a shape memory material.

In some embodiments, the flexible material of the anchor member is sufficiently strong to maintain a flexed configuration transverse to the tissue-penetrating element when pulled proximally with respect to the proximal tissue wall through which the system is extended.

In some embodiments, the distal end of the tissue-penetrating element is sharp to puncture tissue; and the distal tip of the flexible elongate element is extendable distal to the distal end of the distal end of the tissue-penetrating element and soft to engage a tissue wall atraumatically and to protect the tissue wall from the distal end of the tissue-penetrating element. In some embodiments, the tissue-penetrating element has a window formed therethrough, the anchor member extendable through the window to form the anchor feature. In some embodiments, the distal end and window of the tissue-penetrating element are spaced from each other and the distal tip and the anchor member of the flexible elongate element are spaced from each other such that the distal tip extends distally beyond the tissue-penetrating distal end when the anchor member extends from the window.

In some embodiments, the distal tip of the flexible elongate element is soft to engage and to push a tissue wall atraumatically.

In accordance with various principles of the present disclosure, a system for delivering an implantable device to a deployment site includes an access and delivery system and an access and delivery device. The access and delivery system includes a tissue-penetrating element having a distal end configured to penetrate through tissue walls; and an access and delivery device having a proximal portion, an anchor member configured to flex outwardly transverse to the tissue-penetrating element, and a soft distal tip capable of engaging tissue atraumatically. The implantable device delivery and deployment system includes a tissue-penetrating element; and an implantable device. In some embodiments, the anchor member is configured to inhibit distal advancement of the tissue-penetrating element of the implantable device delivery and deployment system; and the soft distal tip is configured to inhibit distal advancement of the tissue-penetrating element of the access and delivery system.

In some embodiments, the soft distal tip is configured to push a tissue wall distally atraumatically to create space for the implantable device at the deployment site. In some embodiments, the distal end of the tissue-penetrating element is sharp to puncture tissue; and the distal tip of the access and delivery device is extendable distal to the distal end of the distal end of the tissue-penetrating element and soft to engage a tissue wall atraumatically and to protect the tissue wall from the distal end of the tissue-penetrating element.

In some embodiments, the anchor member is sufficiently strong to maintain a flexed configuration transverse to the tissue-penetrating element of the access and delivery system when pulled proximally with respect to a proximal tissue wall through which the system is extended.

In some embodiments, the anchor member is sufficiently strong to maintain a flexed configuration transverse to the tissue-penetrating element of the implantable device delivery and deployment system when the tissue-penetrating element of the implantable device delivery and deployment system is advanced toward and against the anchor member, thereby preventing distal advancement of the tissue-penetrating element of the implantable device delivery and deployment system towards the soft distal tip. In some embodiments, the tissue-penetrating element of the implantable device delivery and deployment system has a tissue-penetrating end configured to puncture tissue.

In some embodiments, the anchor member is formed of a flexible material different from the material of at least the proximal portion of the access and delivery device, the flexible material configured to flex transversely with respect to the tissue-penetrating element to form an anchor feature of the access and delivery system. In some embodiments, the anchor member is formed of a shape memory material.

In accordance with various principles of the present disclosure, a method of accessing a deployment site for an implantable device and facilitating deployment of an implantable device includes penetrating through tissue wall of a proximal anatomical structure with a tissue-penetrating element; penetrating through a proximal tissue wall of a distal anatomical structure with the tissue-penetrating element; distally advancing an access and delivery device through the tissue-penetrating element to advance a soft distal tip of the access and delivery device distal to a distal end of the tissue-penetrating element; and extending an anchor member of the access and delivery device with respect to the tissue-penetrating element to flex outwardly and transverse to the tissue-penetrating element.

In some embodiments, the method further includes proximally retracting the anchor member with respect to the proximal tissue wall of the distal anatomical structure to move the proximal wall and the distal anatomical structure towards the proximal anatomical structure.

In some embodiments, the method further includes advancing a tissue-penetrating end of an implantable device delivery and deployment system through the tissue wall of the proximal anatomical structure and the proximal tissue wall of the distal anatomical structure; and impeding distal advancement of the tissue-penetrating end of an implantable device delivery and deployment system with the anchor member.

In some embodiments, the method further includes distally advancing the anchor member of the access and delivery device through a window formed in a wall of the tissue-penetrating element.

In some embodiments, the method further includes advancing the soft distal tip of the access and delivery device distally to push the distal tissue wall of the distal anatomical structure atraumatically away from the proximal tissue wall of the distal anatomical structure.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers with a′ added, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a cross-sectional view of an example of an embodiment of an access and delivery device and system, with an implantable device delivery and deployment system, formed in accordance with aspects of the present disclosure.

FIG. 2 illustrates a cross-sectional view of an access and delivery device and system such as illustrated in FIG. 2, but with the access and delivery device in a deployed configuration.

FIG. 3A illustrates an elevational view of an access and delivery device and system such as illustrated in FIG. 1 delivering an access and delivery device.

FIG. 3B illustrates a view similar to the view of FIG. 3A, but with the access and delivery device in a deployed configuration.

FIG. 3C illustrates a view similar to the view of FIG. 3B, but with the access and

delivery device being used to pull a distal non-adherent anatomical structure proximally toward a proximal non-adherent anatomical structure.

FIG. 3D illustrates an elevational view of an access and delivery device and system such as illustrated in FIG. 2, with an implantable device delivery and deployment system such as illustrated in FIG. 1 commencing to deliver an implantable device.

FIG. 4A illustrates an example of an embodiment of an access and delivery device and system formed in accordance with various principles of the present disclosure and in a delivery configuration.

FIG. 4B illustrates an access and delivery device and system as illustrated in FIG. 4A, but in a deployed configuration.

FIG. 5 illustrates an implantable device delivery system, with an example of an embodiment of an access and delivery device and system in a deployed configuration.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

Various medical procedures involve accessing a distal non-adherent anatomical structure from a proximal non-adherent anatomical structure. The non-adherent anatomical structures may be organs, vessels, muscles, etc., formed by a tissue wall surrounding or defining an anatomical lumen, passage, cavity, etc., within the structure. It will be appreciated that reference herein to an anatomical or body passage, etc., includes naturally-existing passages (e.g., the jejunum) as well as medically-created passages (e.g., a passage created with the use of a medical instrument, such as between a stomach and jejunum), abnormal passages or structures (e.g., formed to a diseased state), or otherwise. In some instances, the non-adherent anatomical structures are held in apposition with respect to each other. In some instances, a device is deployed across the non-adherent anatomical structures and may hold the non-adherent anatomical structures in apposition with respect to each other. It will be appreciated that references herein to non-adherent structures are by way of example and not limitation, as the present disclosure need not be so limited and may apply, as well, to anatomical structures which may adhere to each other, at least to some extent.

In accordance with various principles of the present disclosure, an access and delivery device and system are configured to extend across anatomical structures such as, without limitation, tissue structures or tissue walls. The device and system may be configured to deliver a device across or between anatomical structures such as, without limitation, tissue structures or tissue walls. The device and system are configured to access a distal anatomical structure (e.g., a non-adherent anatomical structure) from a proximal anatomical structure (e.g., another non-adherent anatomical structure) to facilitate delivery of an implantable device to the distal anatomical structure. The implantable device may be extended across the anatomical structures, such as to hold the anatomical structures in apposition. In some embodiments, the access and delivery device and system are configured to hold the distal anatomical structure with respect to the proximal anatomical structure. An implantable device may thereby be advanced from the proximal anatomical structure to the distal anatomical structure (e.g., by penetrating the tissue walls of the respective anatomical structures) without the risk of the anatomical structure being pushed away from the proximal anatomical structure as the implantable device is advanced to the distal anatomical structure. Additionally or alternatively, the access and delivery device and system are configured to bring a distal anatomical structure closer to (e.g., into apposition with) a proximal anatomical structure. The medical professionals are thereby enabled to access a target site (e.g., at the distal anatomical structure) from a different anatomical site (e.g., from within the proximal anatomical structure) without the risk of the different sites shifting or moving away from each other (e.g., the distal anatomical structure shifting or moving away from proximal anatomical structure). Additionally or alternatively, imaging (e.g., endoscopic ultrasound imaging), and establishment and maintenance of access to the anatomical structures during device exchanges are thereby enabled. Various accompanying benefits, such as enhancement of the safety profile of the procedure (e.g., an EUS guided procedure), and shortening of the time required to perform the procedure, may be appreciated by those of ordinary skill in the art.

In accordance with various principles of the present disclosure, an access and delivery device and system which access a distal anatomical structure from a proximal anatomical structure may be used to advance a distal anatomical structure (e.g., a tissue wall thereof) proximally over an implantable device to be advanced from the proximal anatomical structure into the distal anatomical structure (e.g., into a cavity therein). Additionally or alternatively, the access and delivery device and system may simply hold the distal anatomical structure sufficiently securely with respect to the proximal anatomical structure from which the device is delivered so that the implantable device may be advanced into the distal anatomical structure without shifting the distal anatomical structure with respect to the proximal anatomical structure (e.g., moving the distal anatomical structure distally away from the proximal anatomical structure). In some embodiments, the implantable device delivery and deployment system is mounted and advanced over the access and delivery device and system.

In accordance with various principles of the present disclosure, an access and delivery device and system configured to access a cavity within a distal anatomical structure are configured to facilitate delivery of an implantable device within such cavity. For instance, the access and delivery device and system may be configured to be extended distally into the distal anatomical structure to hold open an anatomical cavity therein. The access and delivery device may have a flexible distal end or tip which may protect a distal wall of the distal anatomical structure during performance of a procedure within (e.g., insertion of a device within) the distal anatomical structure. For instance, the flexible tip of the access and delivery device may hold a distal wall of a body lumen away from the proximal wall of the body lumen so that insertion of a device therein (e.g., a cutting device for creating an access hole into the distal tissue wall) does not inadvertently encounter the distal wall of the body lumen. Provision of a flexible tip may thereby protect the walls of the distal anatomical structure during performance of a procedure with respect thereto. It will be appreciated that terms such as body/anatomical cavity, passage, lumen, space, etc., may be used interchangeably herein without intent to limit.

Various embodiments of access and delivery devices and systems and associated methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Moreover, certain features in one embodiment may be used across different embodiments and are not necessarily individually labeled when appearing in different embodiments.

Turning now to the drawings, an example of an embodiment of an access and delivery system 100 is illustrated in conjunction with an implantable device delivery and deployment system 1000. In the illustrated example of an embodiment, the access and delivery system 100 extends through the implantable device delivery and deployment system 1000/the implantable device delivery and deployment system 1000 is mounted over the access and delivery system 100. The access and delivery system 100 is configured to access a deployment site for an implantable device 1010 to be delivered and deployed by the implantable device delivery and deployment system 1000. The access and delivery system 100 may be extended through a proximal anatomical structure, such as a proximal non-adherent anatomical structure, and into a distal anatomical structure, such as a distal non-adherent anatomical structure, to access a deployment site in the distal non-adherent anatomical structure. Additionally or alternatively, the access and delivery system 100 is configured to facilitate deployment of the implantable device 1010. Additionally or alternatively, in procedures in which the implantable device 1010 is to be deployed from a proximal anatomical structure to a distal anatomical structure, the access and delivery system 100 is configured to hold the distal anatomical structure with respect to the proximal anatomical structure to facilitate delivery and deployment of the implantable device 1010. Additionally or alternatively, the access and delivery system 100 is configured to protect a distal wall of the distal anatomical structure from components of the implantable device delivery and deployment system 1000.

More particularly, the example of an embodiment of an access and delivery system 100 illustrated in FIG. 1 includes a tissue-penetrating element 110 having a tissue-penetrating end 112 at a distal end 111 thereof. The tissue-penetrating end 112 may be configured to penetrate through tissue walls of the non-adherent anatomical structures to create initial access openings through the non-adherent anatomical structures for delivery of an implantable device 1010 (delivered by the implantable device delivery and deployment system 1000) therethrough. For instance, the tissue-penetrating end 112 may be sufficiently sharp to puncture through tissue walls. An access and delivery device 120 may be in the form of a flexible elongate element extending through a lumen 115 defined through the tissue-penetrating element 110. The access and delivery device 120 may include a guidewire or other element which facilitates delivery of the implantable device 1010.

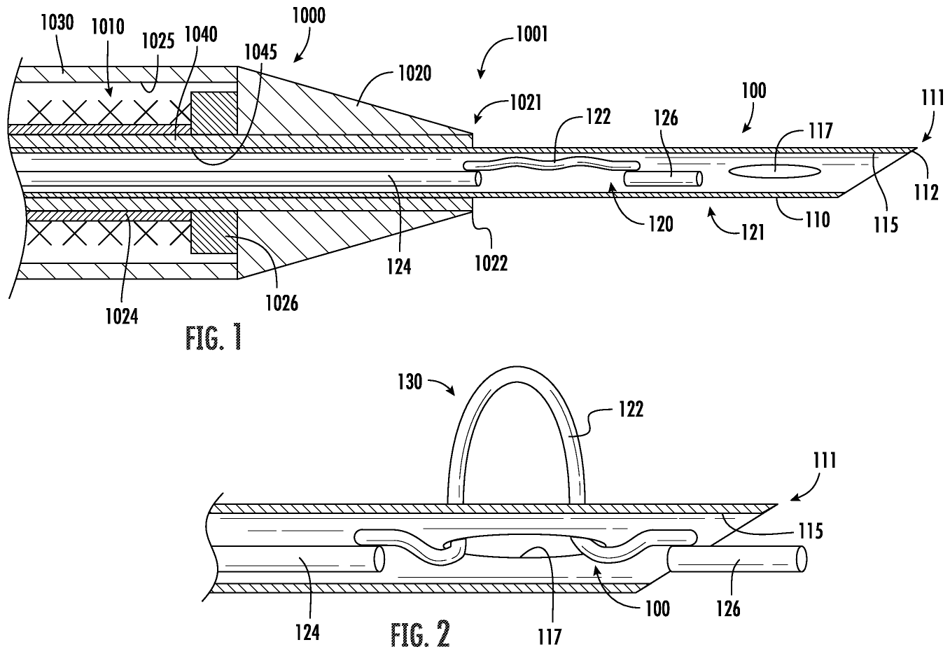

In accordance with various principles of the present disclosure, as illustrated in FIG. 1, the access and delivery device 120 includes an anchor member 122 configured to anchor the access and delivery device 120 with respect to the distal non-adherent anatomical structure with respect to which the implantable device 1010 is to be deployed. The anchor member 122 is formed from a flexible material capable of flexing transversely with respect to the tissue-penetrating element 110 and at least a proximal portion 124 of the access and delivery device 120 to form an anchor feature 130 of the access and delivery device 120 and access and delivery system 100, such as illustrated in FIG. 2. For instance, the anchor member 122 may extend outwardly from the lumen 115 of the tissue-penetrating end 112 through a window 117 formed in the wall of the tissue-penetrating element 110. The anchor member 122 may be formed of a shape memory material (e.g., nitinol, Elgiloy®, etc.) so that distal advancement of the access and delivery device 120 with respect to the tissue-penetrating element 110 to align the anchor member 122 with the window 117 allows the anchor member 122 to flex out the window 117 without further action. The anchor member 122 may be formed of one or more (e.g., two, as shown in the accompanying drawings, or more), flexible elongated elements. In some embodiments, the anchor member 122 is formed by splicing a thin flexible element between the proximal portion 124 and a distal tip 126 of the access and delivery device 120. The anchor member 122, the proximal portion 124, and the soft distal tip 126 of the access and delivery device 120 may be coupled together in any of a variety of manners known to those of ordinary skill in the art, such as welding, adhering with an adhesive, soldering, brazing, or any of a variety of mechanical interfittings (e.g., crimping). In some embodiments, the anchor member 122 is formed by splitting a flexible elongate element, such as a guidewire, into two or more thinner, more flexible elements. It will be appreciated that terms such as flexing, bending, bowing, etc., and other grammatical forms thereof, may be used interchangeably herein without intent to limit.

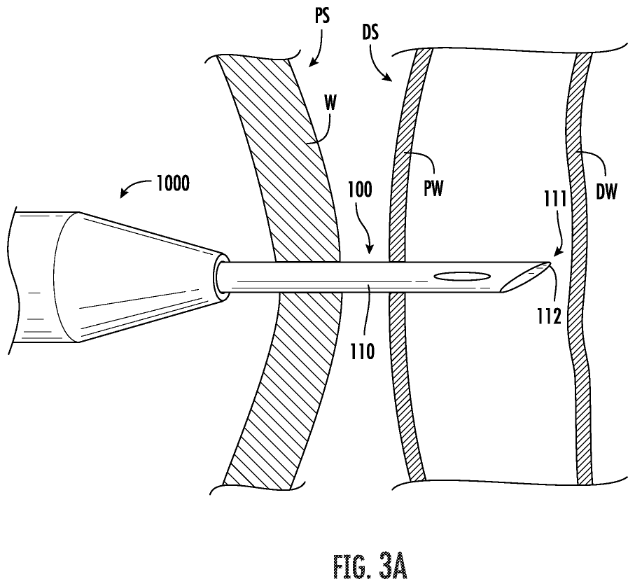

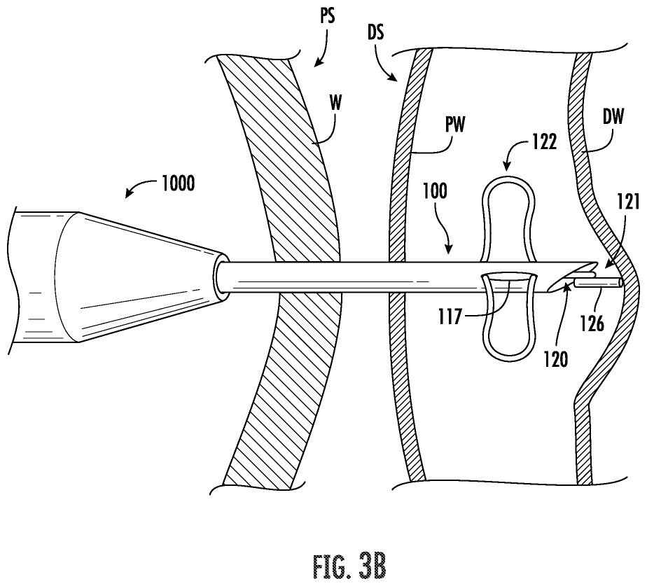

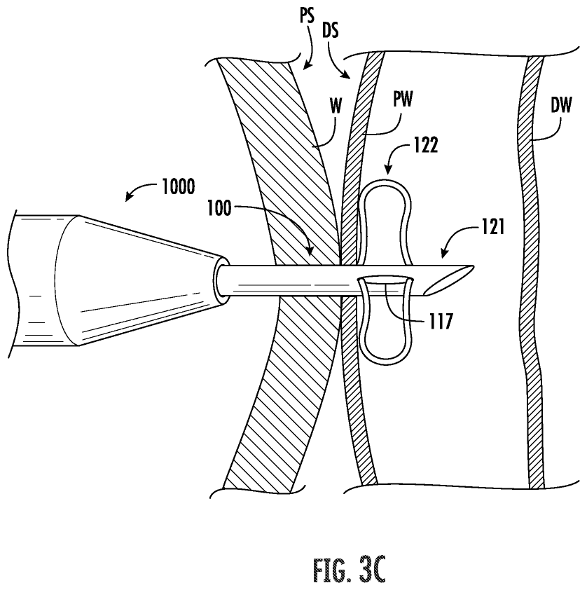

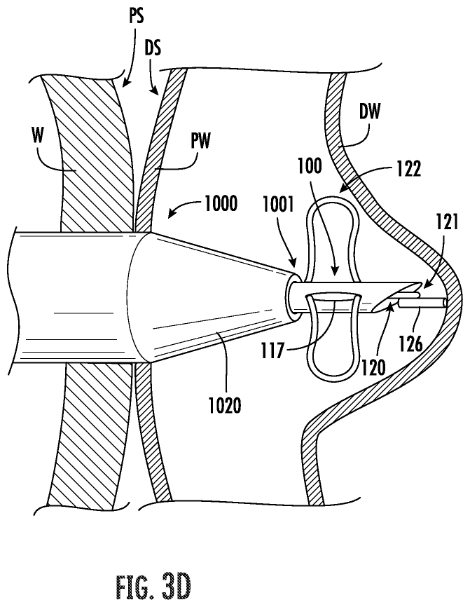

The anchor feature 130 is expanded outwardly with respect to tissue-penetrating element 110 to prevent retraction of the access and delivery device and system 100 once the tissue-penetrating element 110 has been extended through a tissue wall. In the expanded configuration of the anchor member 122, with the anchor feature 130 positioned within a distal non-adherent anatomical structure, the access and delivery device and system 100 may hold the distal non-adherent anatomical structure (in which the anchor feature 130 is positioned) with respect to the proximal non-adherent anatomical structure from which the access and delivery device and system 100 has been advanced into the distal non-adherent anatomical structure. Moreover, the anchor feature 130 may inhibit advancement of other devices into the distal non-adherent anatomical structure, such as to protect the distal tissue wall of the non-adherent anatomical structure in which the anchor feature 130 is positioned. These and other aspects are described in further detail below with reference to FIGS. 3A-3D.

As may be appreciated with reference to FIG. 1 and FIG. 2, the anchor member 122 of the access and delivery device 120 is coupled to or otherwise extends from a proximal portion 124 of the access and delivery device 120. In some embodiments, the distal tip 126 of the access and delivery device 120 is a soft distal tip 126 thin and/or flexible and/or covered with a polymeric coating or layer (to cushion the underlying material) or otherwise formed and/or configured to encounter tissue atraumatically. For instance, the soft distal tip 126 is configured to flex readily upon encountering a tissue wall so that advancement of the access and delivery device 120 against the distal tissue wall of the non-adherent anatomical structure into which the access and delivery system 100 is advanced is atraumatic. In some embodiments, the soft distal tip 126 may be advanced distally once the tissue-penetrating end 112 of the tissue-penetrating element 110 penetrates into an anatomical structure to protect the distal wall of the anatomical structure from the tissue-penetrating end 112. Moreover, the soft distal tip 126 may atraumatically move the distal wall of the non-adherent anatomical structure into which the implantable device 1010 is to be deployed out of the way of the implantable device delivery and deployment system 1000 (e.g., a tissue penetrating end thereof).

During delivery (e.g., transluminal delivery through a patient's body), the access and delivery device 120 remains within the lumen 115 of the tissue-penetrating element 110 with the anchor member 122 proximal to the window 117 in an extended configuration generally aligned with the proximal portion 124. Distal advancement of the access and delivery device 120 with respect to the tissue-penetrating element 110 aligns the anchor member 122 with the window 117. For instance, distal advancement of the access and delivery device 120 to extend the soft distal tip 126 distal to the tissue-penetrating end 112 of the tissue-penetrating element 110 may align the anchor member 122 with the window 117 as well, allowing the anchor member 122 to form the anchor feature 130. In some embodiments, as illustrated in FIG. 2, the tissue-penetrating element 110 and the access and delivery device 120 are configured and dimensioned such that the anchor member 122 is aligned with the window 117 to extend therethrough once the soft distal tip 126 is distal to the tissue-penetrating end 112 of the tissue-penetrating element 110.

An example of a manner in which an access and delivery system 100 formed in accordance with various principles of the present disclosure may be used to access a deployment site for an implantable device 1010 and/or to facilitate delivery of an implantable device 1010, such as with an implantable device delivery and deployment system 1000, is illustrated in FIGS. 3A-3D. The distal end 111 of the tissue-penetrating element 110 is illustrated in FIG. 3A as already advanced distally into a distal non-adherent anatomical structure DS. More particularly, the tissue-penetrating element 110 advances from within the proximal non-adherent anatomical structure PS, through the tissue wall W thereof, distally through the proximal tissue wall PW of the distal non-adherent anatomical structure DS, and into the distal non-adherent anatomical structure DS. As may be appreciated, the tissue-penetrating end 112 of the tissue-penetrating element 110 may be sufficiently sharp to penetrate through the walls of the proximal non-adherent anatomical structure PS and the distal non-adherent anatomical structure DS. The implantable device delivery and deployment system 1000 remains within the proximal non-adherent anatomical structure PS at this stage.

As illustrated in FIG. 3B, the distal end 121 of the access and delivery device 120 is extended distally out the distal end 111 of the tissue-penetrating element 110 preferably before the tissue-penetrating end 112 encounters the distal tissue wall DW of the distal non-adherent anatomical structure DS. The soft distal tip 126 thus extends distal to the tissue-penetrating end 112 of the tissue-penetrating element 110 to protect the distal tissue wall DW from the tissue-penetrating end 112. Optionally, the soft distal tip 126 may extend the distal tissue wall DW away from the proximal tissue wall PW to create a larger space within the distal non-adherent anatomical structure DS to accommodate an implantable device 1010. As also illustrated in FIG. 3B, distal advancement of the access and delivery device 120 aligns the anchor member 122 with the window 117 in the tissue-penetrating element 110 to allow the anchor member 122 to flex outwardly through the window 117 to form the anchor feature 130.

The anchor feature 130 may be sufficiently strong and sized, shaped, configured, and/or dimensioned to allow proximal retraction of the anchor feature 130 to proximally retract the proximal tissue wall PW, as illustrated in FIG. 3C. Proximal retraction of the access and delivery device and system 100 draws the distal non-adherent anatomical structure DS proximally towards the proximal non-adherent anatomical structure PS. The proximal tissue wall PW of the distal non-adherent anatomical structure DS may thereby be brought into apposition with the proximal non-adherent anatomical structure PS to facilitate delivery of the implantable device 1010 through the walls of the proximal non-adherent anatomical structure PS and the distal non-adherent anatomical structure DS. Apposition of the proximal and distal non-adherent anatomical structures PS, DS may be beneficial to prevent leakage of bodily fluids from such structures (e.g., leakage of gastric fluids out of the stomach and into the peritoneum).

Once the proximal and distal non-adherent anatomical structures PS, DS are appropriately positioned with respect to each other, the implantable device delivery and deployment system 1000 may be distally advanced from the proximal non-adherent anatomical structure PS into the distal non-adherent anatomical structure DS, as illustrated in FIG. 3D. As may be appreciated, the anchor feature 130 may impede or prevent further distal advancement of the implantable device delivery and deployment system 1000 which otherwise would result in the distal end 1001 of the implantable device delivery and deployment system 1000 contacting the distal tissue wall DW of the distal non-adherent anatomical structure DS. Such protective function of the anchor feature 130 may be particularly desirable if the implantable device delivery and deployment system 1000 includes a tissue penetrating element 1020, as illustrated in FIG. 3D and FIG. 1.

As may be appreciated with further reference to FIG. 1, the tissue-penetrating element 1020 of the implantable device delivery and deployment system 1000 has a distal end 1021 with a tissue-penetrating end 1022 configured to contact tissue. The tissue-penetrating end 1022 may be sufficiently sharp to puncture tissue, and/or the tissue-penetrating element 1022 may be a cauterization tip with energy supplied thereto by wires 1024 coupled to the tissue-penetrating element 1020 by wiring connections 1026. Once the tissue-penetrating element 1020 has been advanced to the selected deployment site (e.g., within the distal non-adherent anatomical structure DS, between the proximal tissue wall PW and the distal tissue wall DW), the implantable device 1010 may be deployed. In the example of an embodiment of an implantable device delivery and deployment system 1000 illustrated in FIG. 1, the implantable device 1010 is retained within the implantable device delivery and deployment system 1000 by a sheath 1030. The implantable device 1010 fits within a lumen 1035 defined within the sheath 1030 and is constrained therein in a compact delivery configuration capable of being delivered transluminally through a patient's body, such as in a manner known to those of ordinary skill in the art. Once the implantable device delivery and deployment system 1000 is at the deployment site, the sheath 1030 may be proximally withdrawn to allow the implantable device 1010 to shift to an expanded deployment configuration in a variety of manners known to those of ordinary skill in the art. As may be appreciated with reference to FIG. 1, the implantable device 1010 may be delivered within the implantable device delivery and deployment system 1000 over a shaft 1040 having a lumen 1045 defined therein and through which the access and delivery device and system 100 movably (e.g., translatably) extends.

In some embodiments, once the implantable device 1010 has been delivered and deployed, the access and delivery device and system 100 may be retracted proximally to cause the anchor member 122 to return into the lumen 115 of the tissue-penetrating element 110 to allow proximal withdrawal of the access and delivery system 100. In some embodiments, the access and delivery system 100 is retracted into the lumen 1035 of the sheath 1030 of the implantable device delivery and deployment system 1000 for retraction and withdrawal from the patient.

Various modifications may be made to an access and delivery device and system formed in accordance with various principles of the present disclosure without departing from the various principles of the present disclosure. For instance, modifications may be to the tissue-penetrating element or the access and delivery device or both.

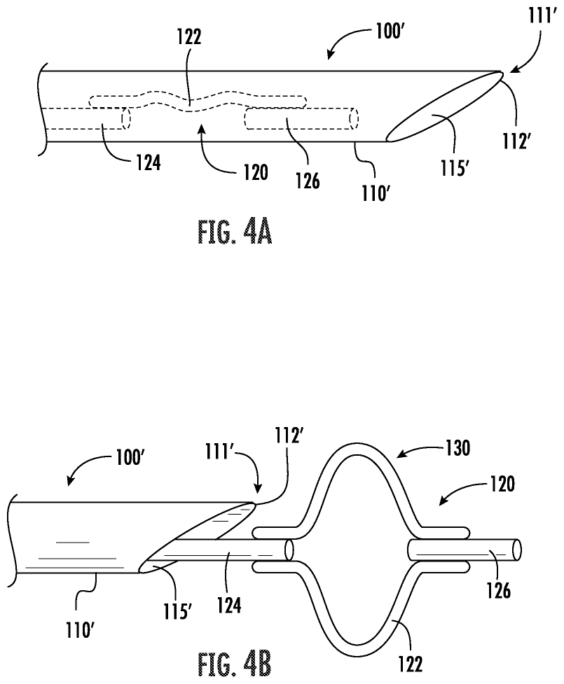

For instance, as illustrated in FIG. 4A and FIG. 4B, an example of an embodiment of an access and delivery system 100′ formed in accordance with various principles of the present disclosure may have a tissue-penetrating element 110′ without a window formed therein. In such embodiment, the anchor member 122 of the access and delivery device 120 is extended distally through the lumen 115′ of the tissue-penetrating element 110′ to extend past the tissue-penetrating end 112′ and out the distal end 111′ of the tissue-penetrating element 110′ to allow the anchor member 122 to shift into an expanded configuration to form the anchor feature 130, as illustrated in FIG. 4B. Various features of the access and delivery device 120 may be the same as the access and delivery device 120 described above with reference to FIG. 1 and FIG. 2, and are labeled with similar reference numbers, reference being made to the above descriptions thereof for the sake of brevity.

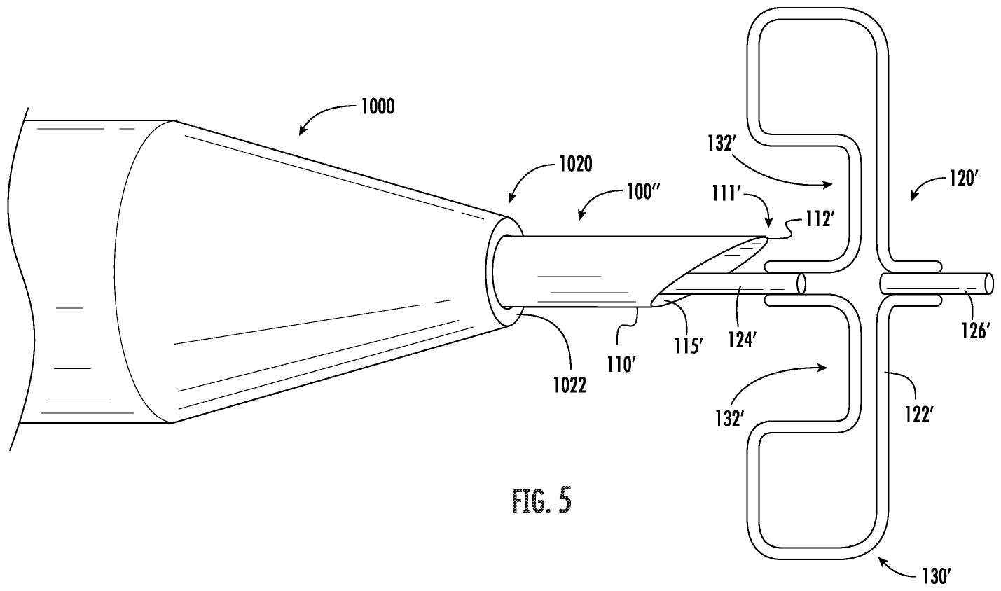

Additionally or alternatively, one or more features or elements of the access and delivery device may be modified. In the example of an embodiment of access and delivery device and system 100″ illustrated in FIG. 5, a modified access and delivery device 120′ is illustrated with the modified tissue-penetrating element 110′ illustrated in FIGS. 4A and 4B. However, it will be appreciated that the modified access and delivery device 120′ may be used with the tissue-penetrating element 110 illustrated in FIG. 1 and FIG. 2 instead. The modified access and delivery device 120′ illustrated in FIG. 5 has a modified anchor member 122′ sized, shaped, configured, and/or dimensioned with respect to the implantable device delivery and deployment system 1000 to be advanced with respect thereto to limit distal advancement of the implantable device delivery and deployment system 1000 with respect to a distal tissue wall. In some embodiments, the illustrated example of an embodiment of an anchor feature 130′ may configured with pockets 132′ sized, shaped, configured, and/or dimensioned to restrain or capture the tissue-penetrating element 1020 of the implantable device delivery and deployment system 1000 to impede further distal advancement of the tissue-penetrating element 1020. The tissue-penetrating end 1022 may thereby be prevented from contacting (and potentially damaging) the distal wall of the non-adherent anatomical structure into which the implantable device delivery and deployment system 1000 is advanced. The modified access and delivery device 120′ may also include a soft distal tip 126′ and a proximal portion 124′ similar to the soft distal tip 126 and proximal portion 124 of the access and delivery device 120 described above with reference to FIG. 1 and FIG. 2, reference being made to the above descriptions thereof for the sake of brevity.

It is to be understood by one of ordinary skill in the art that the present discussion is a description of illustrative examples of embodiments only, and is not intended as limiting the broader aspects of the present disclosure. It will be appreciated that various examples of embodiments described above can be arranged and operate in substantially the same or similar manners. Accordingly, for the sake of brevity and convenience, and without intent to limit, common elements with common functions are indicated with the similar reference characters (with or without a′ thereafter), reference being made to prior descriptions of similar elements and operations.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of a devices, systems, and methods such as described above, in addition to those discussed above, may be appreciated by those of ordinary skill in the art.

Embodiments of the present disclosure may be used with procedures for treating the gastrointestinal system, or other anatomical systems such as in the abdominal cavity, digestive system, urinary tract, reproductive tract, respiratory system, cardiovascular system, circulatory system, and the like. The medical devices described herein may be delivered with additional medical devices for navigating body lumens, including, for example, catheters, endoscopes, duodenoscopes, ureteroscopes, bronchoscopes, colonoscopes, arthroscopes, cystoscopes, hysteroscopes, laparoscopes, and the like. The disclosed medical devices and systems may also be inserted via different access points and approaches, e.g., percutaneously, endoscopically, laparoscopically, or combinations thereof. In view of the above descriptions, it will be appreciated that the devices, systems, and methods disclosed herein can be used to form one or more anastomoses, and can be used with basic endoscopic tools, catheters, laparoscopes, general surgery tools, etc. For example, the implantable device delivery and deployment system described herein may be a catheter-based stent delivery device which can be used with an endoscope to form one anastomosis, for example between two portions of the intestines. An endoscopic-based device could be used to form an anastomosis between the fundal pouch and a portion of the intestines, such as the jejunum. A combination of a laparoscopic-based device and a catheter-device as described herein could also be used to form a single anastomosis.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An access and delivery device and system configured to access a deployment site beyond a proximal tissue wall of an anatomical structure, said access and delivery device and system comprising:
a tissue-penetrating element having a distal end configured to penetrate through tissue walls; and
an access and delivery device having a proximal portion, an anchor member, and a distal tip; wherein:
said anchor member is formed of a flexible material different from the material of at least said proximal portion of said flexible elongate element, said flexible material configured to flex transversely with respect to said tissue-penetrating element to form an anchor feature of said system.

2. The system of claim 1, wherein said anchor member is formed of a shape memory material.

3. The system of claim 1, wherein the flexible material of said anchor member is sufficiently strong to maintain a flexed configuration transverse to said tissue-penetrating element when pulled proximally with respect to the proximal tissue wall through which said system is extended.

4. The system of claim 1, wherein
said distal end of said tissue-penetrating element is sharp to puncture tissue; and
said distal tip of said flexible elongate element is extendable distal to said distal end of said distal end of said tissue-penetrating element and soft to engage a tissue wall atraumatically and to protect the tissue wall from said distal end of said tissue-penetrating element.

5. The system of claim 4, wherein said tissue-penetrating element has a window formed therethrough, said anchor member extendable through the window to form said anchor feature.

6. The system of claim 5, wherein said distal end and window of said tissue-penetrating element are spaced from each other and said distal tip and said anchor member of said flexible elongate element are spaced from each other such that said distal tip extends distally beyond said tissue-penetrating distal end when said anchor member extends from the window.

7. The system of claim 1, wherein said distal tip of said flexible elongate element is soft to engage and to push a tissue wall atraumatically.

8. A system for delivering an implantable device to a deployment site, said system comprising:
an access and delivery system comprising:
a tissue-penetrating element having a distal end configured to penetrate through tissue walls; and
an access and delivery device having a proximal portion, an anchor member configured to flex outwardly transverse to said tissue-penetrating element, and a soft distal tip capable of engaging tissue atraumatically; and
an implantable device delivery and deployment system comprising:
a tissue-penetrating element; and
an implantable device;
wherein:
said anchor member is configured to inhibit distal advancement of said tissue-penetrating element of said implantable device delivery and deployment system; and
said soft distal tip is configured to inhibit distal advancement of said tissue-penetrating element of said access and delivery system.

9. The system of claim 8, wherein said soft distal tip is configured to push a tissue wall distally atraumatically to create space for said implantable device at the deployment site.

10. The system of claim 9, wherein:
said distal end of said tissue-penetrating element is sharp to puncture tissue; and
said distal tip of said access and delivery device is extendable distal to said distal end of said distal end of said tissue-penetrating element and soft to engage a tissue wall atraumatically and to protect the tissue wall from said distal end of said tissue-penetrating element.

11. The system of claim 8, wherein said anchor member is sufficiently strong to maintain a flexed configuration transverse to said tissue-penetrating element of said access and delivery system when pulled proximally with respect to a proximal tissue wall through which said system is extended.

12. The system of claim 8, wherein said anchor member is sufficiently strong to maintain a flexed configuration transverse to said tissue-penetrating element of said implantable device delivery and deployment system when said tissue-penetrating element of said implantable device delivery and deployment system is advanced toward and against said anchor member, thereby preventing distal advancement of said tissue-penetrating element of said implantable device delivery and deployment system towards said soft distal tip.

13. The system of claim 11, wherein said tissue-penetrating element of said implantable device delivery and deployment system has a tissue-penetrating end configured to puncture tissue.

14. The system of claim 8, wherein said anchor member is formed of a flexible material different from the material of at least said proximal portion of said access and delivery device, said flexible material configured to flex transversely with respect to said tissue-penetrating element to form an anchor feature of said access and delivery system.

15. The system of claim 13, wherein said anchor member is formed of a shape memory material.

16. A method of accessing a deployment site for an implantable device and facilitating deployment of an implantable device, said method comprising:
penetrating through tissue wall of a proximal anatomical structure with a tissue-penetrating element;
penetrating through a proximal tissue wall of a distal anatomical structure with the tissue-penetrating element;
distally advancing an access and delivery device through the tissue-penetrating element to advance a soft distal tip of the access and delivery device distal to a distal end of the tissue-penetrating element; and
extending an anchor member of the access and delivery device with respect to the tissue-penetrating element to flex outwardly and transverse to the tissue-penetrating element.

17. The method of claim 16, further comprising proximally retracting the anchor member with respect to the proximal tissue wall of the distal anatomical structure to move the proximal wall and the distal anatomical structure towards the proximal anatomical structure.

18. The method of claim 16, further comprising:
advancing a tissue-penetrating end of an implantable device delivery and deployment system through the tissue wall of the proximal anatomical structure and the proximal tissue wall of the distal anatomical structure; and
impeding distal advancement of the tissue-penetrating end of an implantable device delivery and deployment system with the anchor member.

19. The method of claim 16, further comprising distally advancing the anchor member of the access and delivery device through a window formed in a wall of the tissue-penetrating element.

20. The method of claim 16, further comprising advancing the soft distal tip of the access and delivery device distally to push the distal tissue wall of the distal anatomical structure atraumatically away from the proximal tissue wall of the distal anatomical structure.
